(19) United States
(12) Patent Application Publication
(10) Pub. No.: US 2019/0242938 A1
(43) Pub. Date: Aug. 8, 2019

(54) METHODS OF BANDGAP ANALYSIS AND MODELING FOR HIGH K METAL GATE

(71) Applicants: GLOBALFOUNDRIES Inc., Grand Cayman (KY); KLA-Tencor, Milpitas, CA (US)

(72) Inventors: Min DAI, Allendale, NJ (US); Dominic SCHEPIS, Wappingers Falls, NY (US); Qiang ZHAO, Milpitas, CA (US); Ming DI, Pleasanton, CA (US); Dawei HU, Pleasanton, CA (US)

(73) Assignees: GLOBALFOUNDRIES Inc., Grand Cayman (KY); KLA-Tencor, Milpitas, CA (US)

(21) Appl. No.: 15/887,357

(22) Filed: Feb. 2, 2018

(51) Int. Cl. G01R 31/265; H01L 21/66; H01L 21/28; G01R 31/02; G01R 31/26; G01B 15/02; G01N 21/21; G01N 21/33; G01N 33/00

(52) U.S. Cl. CPC G01R 31/2656; H01L 22/12; H01L 21/28202; H01L 21/28185; G01R 31/025; H01L 29/42364; G01B 15/02; G01N 21/211; G01N 21/33; G01N 33/00; G01R 31/2601

(57) ABSTRACT

Methods of precisely analyzing and modeling band gap energies and electrical properties of a thin film are provided. One method includes: obtaining a substrate and a thin film disposed above the substrate, the thin film including an interfacial layer above the substrate, and a high-k layer above the interfacial layer; determining a thickness of the thin film; analyzing the thin film using deep ultraviolet spectroscopy ellipsometry to determine the photon energy of reflected light; using a model to determine a set of bandgap energies extracted from a set of results of the photon energy of the analyzing step; and determining at least one of: a leakage current from a main bandgap energy, a nitrogen content from a sub bandgap energy, and an equivalent oxide thickness from the nitrogen content and a composition of the interfacial layer.

### FIELD OF THE INVENTION

The present invention relates to methods of analyzing the bandgap of a high-k metal gate, and more particularly, to better modeling of the bandgap to determine electrical parameters.

### BACKGROUND

For complementary metal-oxide-semiconductor (CMOS) devices at or below 45 nm, equivalent oxide thickness (EOT) scaling and control has become critical, especially with the use of high-k/metal gate (HKMG) technology. In particular, a dielectric with higher dielectric constant than SiO₂ and metal gate are used, with some material selection and combinations for work function tuning necessary. To further scale the EOT of HKMG, nitridation to the interfacial layer and/or high-k dielectric are often introduced, with nitridation possible. However, control of the leakage current of the high-k gate stack is important, which is determined frequently by the elemental profile (such as nitrogen) and composition of the layers of the device. There is a great challenge in linking the physical thickness or the composition to the electrical properties using previous techniques due to the complexities. For instance, inline X-ray photoelectron spectroscopy (XPS) has been used to determine the film composition, such as the nitrogen concentration, but the throughput is inherently slow and its penetration depth is limited, and no other film electrical properties can be provided. Other compositional measurement techniques, such as offline secondary ion mass spectrometry (SIMS), can be destructive and extremely slow. No electrical properties can be obtained either by SIMS, XPS, or other previous measurement techniques. Additionally, studying the electrical parameters typically requires the full building of end transistor devices in order to measure and quantify results, which may take two to four months.

Therefore, it may be desirable to develop methods of analyzing physical parameters which can reliably model and predict electrical parameters of a thin film in a single environment, without requiring building of an end functional device.

### BRIEF SUMMARY

The shortcomings of the prior art are overcome and additional advantages are provided through the provisions, in one aspect, a method that includes, for instance: obtaining a substrate and a thin film disposed above the substrate, the thin film including an interfacial layer above the substrate, and a high-k layer above the interfacial layer; determining a thickness of the thin film; analyzing the thin film using deep ultraviolet spectroscopy ellipsometry to determine the photon energy of reflected light; using a model to determine a set of bandgap energies extracted from a set of results of the photon energy of the analyzing step; and determining a leakage current from a main bandgap energy of said set of bandgap energies.

In another aspect, a method includes, for instance: obtaining a substrate having a thin film disposed above the substrate, the thin film including an interfacial layer above the substrate, and a high-k layer above the interfacial layer; determining a thickness of the thin film; analyzing the thin film using deep ultraviolet spectroscopy ellipsometry to determine the photon energy of reflected light; using a model to determine a set of bandgap energies extracted from the photon energy of a set of results of the analyzing step; and determining a nitrogen content from a sub bandgap energy of the set of bandgap energies.

In another aspect, an equivalent oxide thickness of a thin film can be determined from the nitrogen content and a composition of the interfacial layer.

In another aspect, a method includes, for instance: obtaining a substrate and a thin film disposed above the substrate, the thin film including an interfacial layer directly above the substrate, and a high-k layer directly above the interfacial layer; determining a thickness of the thin film; analyzing the thin film by using deep ultraviolet spectroscopy ellipsometry to cause reflected light and determining photon energy of the reflected light; using a model to extract a set of bandgap energies from a set of results of the determined photon energy of the reflected light; and determining a property of the thin film using at least one of said set of bandgap energies

### BRIEF DESCRIPTION OF THE SEVERAL VIEWS OF THE DRAWINGS

One or more aspects of the present invention are particularly pointed out and distinctly claimed as examples in the claims at the conclusion of the specification. The foregoing and other objects, features, and advantages of the invention are apparent from the following detailed description taken in conjunction with the accompanying drawings in which:

FIG. 1 depicts one embodiment of a method of analyzing a thin film, in accordance with one or more aspects of the present invention;

FIG. 2 depicts a cross-sectional elevation view of one embodiment of an intermediate semiconductor structure having a substrate and a thin film including an interfacial layer and a high-k layer, in accordance with one or more aspects of the present invention;

FIG. 3 depicts an ellipsometer, in accordance with one or more aspects of the present invention;

FIG. 4 depicts a sample data set for determining a bandgap energy, in accordance with one or more aspects of the present invention;

FIG. 5 depicts a sample data set for determining a set of bandgap energies, in accordance with one or more aspects of the present invention;

FIG. 6 depicts a linear correlation of a bandgap energy for determining leakage current, in accordance with one or more aspects of the present invention;

FIG. 7 depicts a linear correlation of a bandgap energy for determining nitrogen content, in accordance with one or more aspects of the present invention; and

FIG. 8 depicts a linear correlation of a bandgap energy for determining the EOT, in accordance with one or more aspects of the present invention.

### DETAILED DESCRIPTION

Aspects of the present invention and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the invention in detail. It should be understood, however, that the detailed description and the specific examples, while indicating embodiments of the invention, are given by way of illustration only, and are not by way of limitation. Various substitutions, modifications, additions and/or arrangements within the spirit and/or scope of the underlying inventive concepts will be apparent to those skilled in the art from this disclosure. Note also that reference is made below to the drawings, which are not drawn to scale for ease of understanding, wherein the same reference numbers used throughout different figures designate the same or similar components.

Generally stated, disclosed herein are methods of analyzing a thin film. Advantageously, the methods allow for determining physical and electrical properties of the thin film without requiring testing of a finished device.

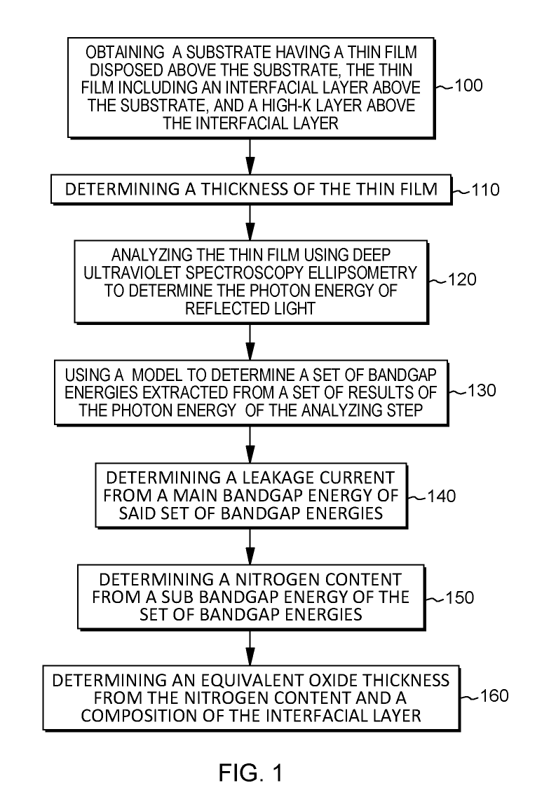

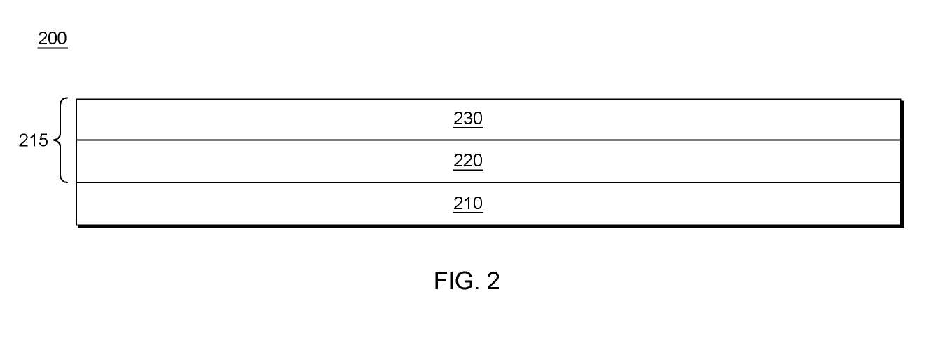

In one aspect, in one embodiment, as shown in FIG. 1, a method of analyzing a thin film may include obtaining an intermediate semiconductor device 100. An example semiconductor device is depicted in FIG. 2. For instance, the intermediate semiconductor device 200 can include a substrate 210, which may include silicon, silicon germanium, and silicon carbide, for instance. Above the substrate 210 may be disposed a thin film, such as a dielectric film 215, including an interfacial layer 220. The interfacial layer 220 can include any material, such as SiO₂ or SiON, which assists further layers with interfacing effectively to substrate 210. With the scaling of equivalent oxide thickness (EOT) of such films, interfacial layer (IL) 220 is often introduced in order to facilitate use of, for instance, high-k layer 230 disposed thereon as part of the thin film 215. While described as a high-k layer, it should be understood that this could also include HfO₂ and other hafnium based layers, such as HfSiOₓ, any dielectric film, other high-k materials such as SiO2, SiₓNy, SiON, La₂O₃ and Al₂O₃, and in some instances, a metal gate material such as TiN. Any or all of the layers of intermediate semiconductor 200 may be nitrogenized (or nitridated) and can include other elements useful in an end device, including but not limited to La and TiAlC.

The dielectric constant, or k value, of the thin film, or stack, of an intermediate semiconductor device 200 is determined by the elemental profile and composition of the films thereon. However, measuring the thickness and composition of the stack typically requires first building the end device, and then multiple testing procedures and environments, such as multiple different spectroscopic ellipsometry (SE) technologies combined with X-ray photoelectron spectroscopy (XPS). However, as discussed above, these methods are slow and often inaccurate.

Returning to FIG. 1, according to some embodiments, a thickness of the thin film of the intermediate semiconductor device 200 (FIG. 2) may be determined 110, for instance, either using the XPS methods described above, or alternatively, by controlling the deposition to engineer the layers to a controlled and desired thickness. The thin film 215 can then be analyzed using deep ultraviolet spectroscopy ellipsometry (DUVSE) 120. This technique is more effective than previous methods of analysis. However, in order to determine a set of bandgap energies 130, an effective linear transgression model is used to extract from a set of results from the analyzing step 120 a set of bandgap energies. In some embodiments, a Bruggeman effective model approximation method can be used. This will be described in conjunction with the use of a DUVSE according to certain embodiments below.

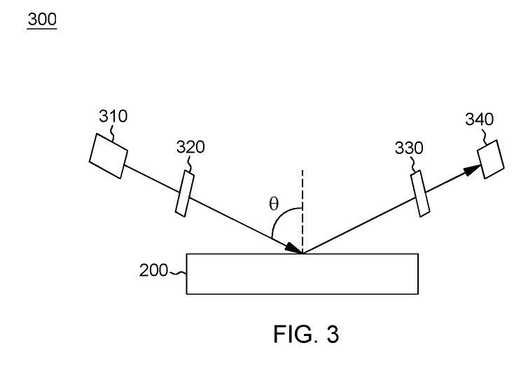

Turning to FIG. 3, an ellipsometer 300 is depicted which is capable of DUVSE analysis of thin film 215 (FIG. 2). Spectroscopic Ellipsometry (SE) technologies, especially DUVSE, can be used for in-situ non-invasive analysis methods suitable for thin film thickness and refractive index measurement necessary for analysing intermediate semiconductor device 200. In order to analyse the layers 220 and 230, using the substrate 210 as background, the band gap is investigated using DUVSE technology (approximately 150 nm to approximately 800 nm wavelength range) and band gap value is extracted from the thin film stack dispersion curve which are the real, ε1, and the imaginary, ε2, part of the dielectric function (or refractive index, n, and extinction coefficient, k) from the SE measurements. Any high performance film metrology tool with DUVSE capability that meets the tight process tolerances required for thickness, refractive index, and composition measurements on critical films can be utilized.

An ellipsometer 300 is designed to measure intensity ratio and phase shift (tan Ψ and cos Δ) between S polarized light and P polarized light. The basic optical path is shown in FIG. 3. For instance, broadband spectroscopic unpolarized light (150 nm˜800 nm) can be generated by the light source 310. Linear polarized light can be obtained after passing through the polarizer 320 and the light interacts with the layer stack on the intermediate semiconductor device 200. Following this, the linear light can become elliptically polarized light carrying intensity ratio and phase shift (tan Ψ and cos Δ) information, which are sensitive to the refractive index and thickness of the films in the stack. Next, the elliptically polarized light can pass through an analyser 330 and become linear polarized light again. Lastly, the detector 340 can receive the linear polarized light signal. The value tan Ψ and cos Δ can be extracted as a function of wavelength. The complex refractive index, complex dielectric function, and thickness of the film can be extracted from the measured tan Ψ and cos Δ curve. In order to measure the thickness of high-k films accurately, the optical properties of high-k films have to be described accurately by the dispersion models, or effective models, used to represent the films.

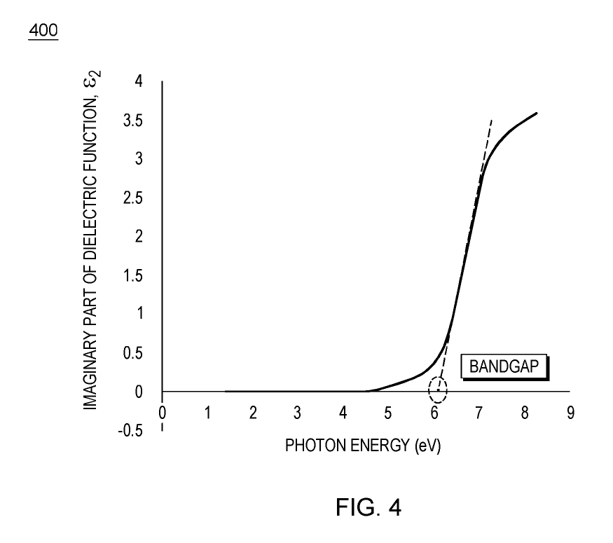

A number of effective models have been used in the SE for process monitoring previously. In some embodiments, a Bruggeman Effective Model Approximation (BEMA) model with one layer method is developed for the film stack depicted in FIG. 2. Using BEMA, the high-k layer 230 and the IL 220 were treated as single layer and thus combined with four sub-components (SiO₂, HfO₂ with or without nitridation, SiN, and Si) which are present in the high-k layer 230 and the IL 220. In order to obtain the dispersion curve plot, both fraction parameters of sub component in BEMA model and thickness parameter of intermediate semiconductor device 200 can be extracted from measurements of raw data tan Ψ and cos Δ. Following this extraction, a linear fit function is applied in the imaginary part of dielectric function dispersion curve within a certain wavelength range to extract band gap value. The band gap value can be accurately determined by linear fit line extrapolation to zero as depicted in FIG. 4.

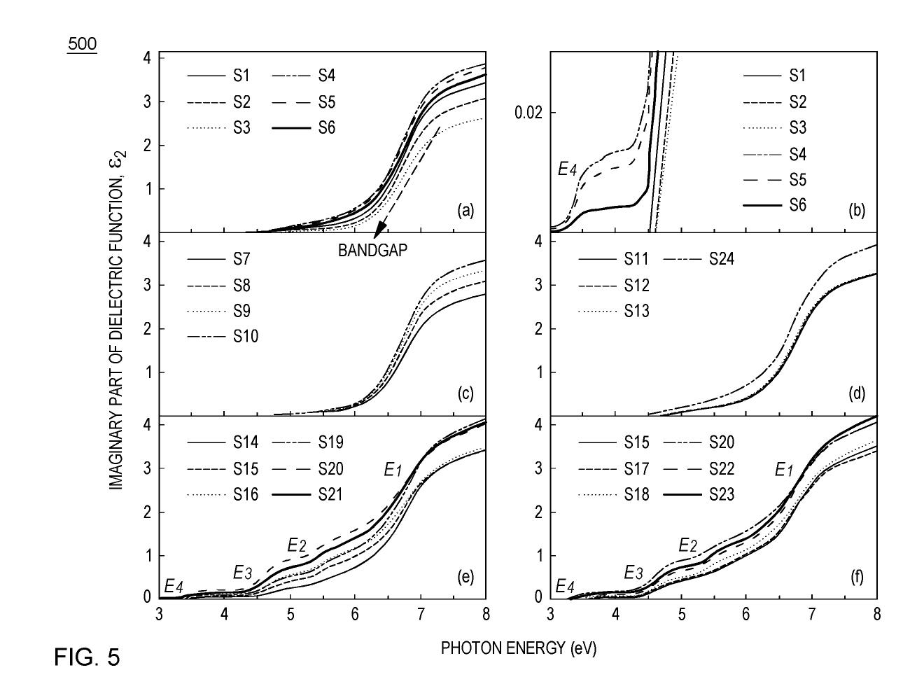

Similarly, using this information, as depicted in FIG. 5, a set of results 500 for different samples of intermediate semiconductor device 200 (FIG. 2) can be used to determine the set of bandgap energies. As can be seen, the set of bandgap energies can be and can include a main bandgap energy (E₁), as well as up to three sub bandgap energies (E₂-E₄), as indicated in FIG. 5 (e) and (f). The sub bandgap state E₂ appears to be the result of HfO₂ crystallization during annealing, while the sub bandgap states E₃ and E₄ were previously unobserved prior to methods according to current embodiments. The sub bandgap energy E₃ only appears after nitridation and anneal together as suggested by comparing FIG. 5 (d) and FIG. 5 (e), so it is likely the result of defect states corresponding to nitrogen movement inside the film during the annealing. The sub bandgap energy E₄ is observed whenever nitrogen exists, regardless of the IL 220 (FIG. 2). Thus, the sub bandgap energy E₄ corresponds to the nitrogen related states in the bandgap. Accordingly, it can be utilized to quantify the total nitrogen concentration inside the high-k/IL stacks 230 and 220. The different lines in these figures correspond to known samples and show the comparison of the bandgaps E1-E4 in known samples.

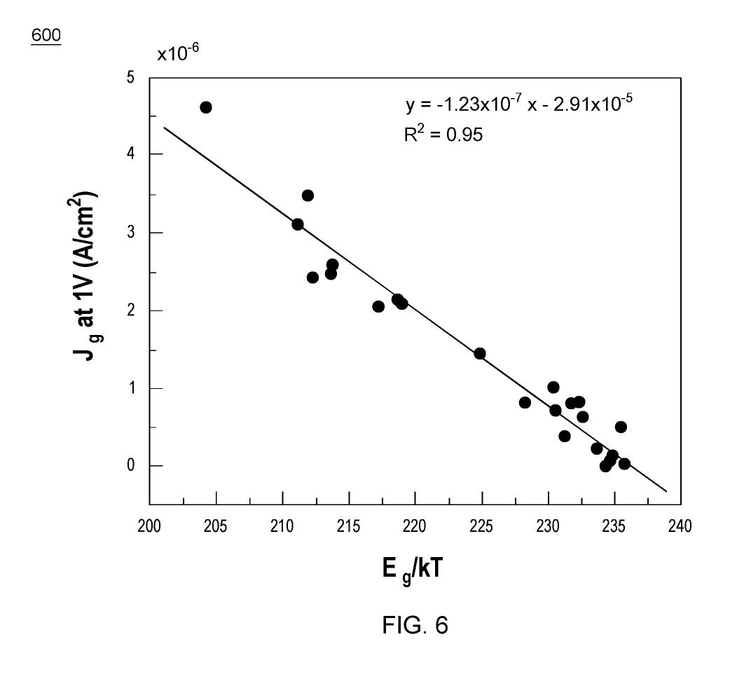

Thus, according to some embodiments, the main bandgap energy can be used to determine the leakage current of intermediate semiconductor device 200, as depicted at 140 in FIG. 1. For instance, by measuring a current applied to the device at 1 V (J_{g}), where J_{g} as the function of E_{g}/kT, in some embodiments there is a 1^{st} order linear correlation between J_{g} and E_{g} of the main bandgap energy E₁, where the kT is approximately 26 mV, E_{g} representing the bandgap energy and kT representing the thermal energy. In some embodiments, the linear function derived can have a coefficient of determination, R², of approximately 0.95, for instance of a linear equation of approximately y=1.23×10⁻⁷x−2.91×10⁻⁵. For instance, as seen in FIG. 6, the linear function is illustrated in a set of results 600 showing the ability to determine the leakage current. This allows for methods according to certain embodiments to be generic and reliably accurate for predicting the current leakage of an end device from the linear function, as seen in FIG. 6, utilizing intermediate semiconductor device 200, but without needing to test the end device. Thus, according to some embodiments, if the leakage current is not within a predetermined threshold, intermediate semiconductor device 200 may not be used for a device, or it may be used in a different device than originally planned based on the leakage current of the stack.

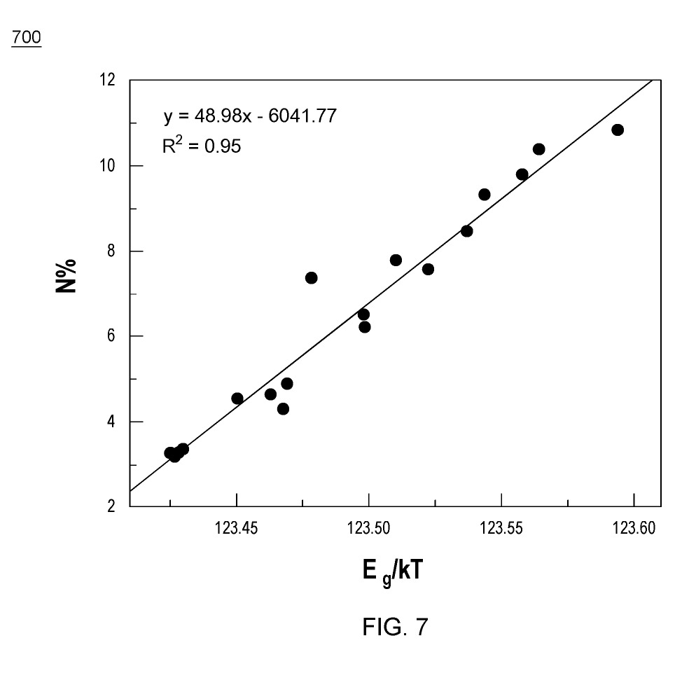

In another embodiment, a sub bandgap energy, E₄, may be used to determine a nitrogen content, or N %. For instance, N % can be determined as the linear function of E_{g}/kT, in some embodiments where kT is approximately 26 mV. Accordingly, N % can be linearly correlated to E_{g} of the sub bandgap energy E₄, in some embodiments with a coefficient of determination, R², of approximately 0.95, for example for a linear equation of approximately y=48.98x−6041.77. For instance, as seen in FIG. 7, the linear function is illustrated in a set of results 700 showing the ability to determine the N %. As such, N % can be directly characterized by the measurement of the sub bandgap energy state E₄ without requiring the complicated modeling in SE measurements previously necessary, and at the same time and in the same environment as determining the leakage current as described above utilizing the ellipsometer 300 of FIG. 3.

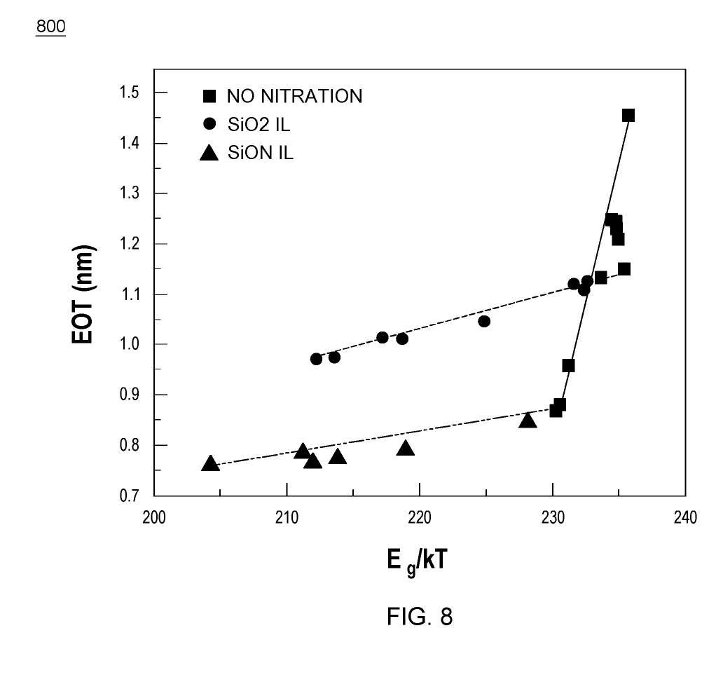

In further embodiments, an equivalent oxide thickness (EOT) can be determined 160 (FIG. 1), from the nitrogen content, which can be determined or known, and a composition of the IL 220. As seen in FIG. 8, a set of results 800 can be used to accurately predict the EOT, but the N % and composition of the IL 220 must be known in order to make this determination, according to some embodiments. In some embodiments, the EOT is less than 1.5 nm, and in further embodiments, may be less than Inm.

According to some embodiments, ultra-fast methods of analyzing physical parameters which can reliably model and predict electrical parameters of a thin film without requiring building of an end functional device have been developed. By using a highly sensitive deep UV SE technology (wavelength range of 150 nm-800 nm) and Bruggeman effective model approximation method (BEMA), HfO₂ on SiO₂ and SiON IL stacks with different thickness and nitrogen concentration with ultra low EOT (<1.5 nm) were studied. The derived imaginary part of the dielectric function was then analyzed to extract the value of band gap energy (E_{g}) including multiple sub band gap states. Based on these, a new sub band gap state was found which can be directly correlated to the total nitrogen concentration in the film. A direct linear correlation (R²=0.95) was found between the nitrogen concentration and E_{g} of the corresponding sub band gap states. The calculated main band gap energy was quantitatively correlated, for the first time, to the leakage current of a final device built on the same film with R²=0.95. Therefore, these correlations suggest the application of optical SE technique at deep UV range can be extended for fast and accurate characterization of HKMG composition and film leakage previously un-discovered.

Thus, according to embodiments, by using deep DUVSE analysis methods to determine the optical band gap energy of high-k/IL stacks 230 and 220, in some embodiments for high-k/metal gate CMOS with EOT of less than 1.5 nm. Utilizing sub bandgap and main bandgap energies, which correspond to the existence or movement of nitrogen inside the films and a tunneling current of the films, the band gap energies are found to be linearly correlated to the HfO₂/IL films' leakage current and N % (R²=0.95). This correlation allows for predicting the electrical and physical properties above using DVSE without the need of complicated physical analysis (e. g. XPS, SIMS) or electrical measurement on fully built devices, but for modeling based upon the intermediate semiconductor device 200 and accurate predictions based on the bandgap energies.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A method of analyzing a thin film, the method comprising:
obtaining a substrate and a thin film disposed above the substrate, the thin film including an interfacial layer above the substrate, and a high-k layer above the interfacial layer;
determining a thickness of the thin film;
analyzing the thin film using deep ultraviolet spectroscopy ellipsometry to determine the photon energy of reflected light;
using a model to determine a set of bandgap energies extracted from a set of results of the photon energy of the analyzing step; and
determining a leakage current from a main bandgap energy of said set of bandgap energies.

2. The method of claim 1, wherein the interfacial layer is chosen from a group consisting of: SiO₂ and SiON.

3. The method of claim 1, wherein the thickness is determined by one of: X-ray photoelectron spectroscopy or deriving from a controlled deposition when the thin film is formed to be disposed above the substrate.

4. The method of claim 1, wherein the model is a Bruggeman effective model approximation method.

5. The method of claim 1, wherein the leakage current is determined by a function of a linear correlation of E_{g}/kT for the main bandgap energy, wherein kT is a thermal energy and is approximately 26 mV and E_{g} is the main bandgap energy.

6. The method of claim 5, wherein a coefficient of determination of the linear correlation is approximately 0.95 for a linear equation of approximately y=1.23×10⁻⁷x−2.91×10⁻⁵.

7. The method of claim 1, wherein the thin film is used as a part of a high-k/metal gate.

8. The method of claim 1, wherein the main bandgap energy is an inherent bandgap of the thin film and is always present.

9. A method of analyzing a thin film, the method comprising:
obtaining a substrate and a thin film disposed above the substrate, the thin film including an interfacial layer above the substrate, and a high-k layer above the interfacial layer;
determining a thickness of the thin film;
analyzing the thin film using deep ultraviolet spectroscopy ellipsometry to determine the photon energy of reflected light;
using a model to determine a set of bandgap energies extracted from the photon energy of a set of results of the analyzing step; and
determining a nitrogen content from a sub bandgap energy of the set of bandgap energies.

10. The method of claim 9, wherein the interfacial layer is chosen from a group consisting of: SiO₂ and SiON.

11. The method of claim 9, wherein the thickness is determined by one of: X-ray photoelectron spectroscopy or deriving from a controlled deposition when the thin film is formed to be disposed above the substrate.

12. The method of claim 9, wherein the model comprises a Bruggeman effective model approximation method.

13. The method of claim 9, wherein the nitrogen content is a function of a linear correlation of E_{g}/kT for the sub bandgap energy, wherein kT is a thermal energy and is approximately 26 mV and E_{g} is the sub bandgap energy

14. The method of claim 13, wherein a coefficient of determination of the linear correlation is approximately 0.95 for a linear equation of approximately y=48.98x−6041.77.

15. The method of claim 9, further comprising:
determining an equivalent oxide thickness of the interfacial layer from the nitrogen content and a composition of the interfacial layer.

16. The method of claim 9, wherein the thin film is used as a part of a high-k/metal gate.

17. The method of claim 9, wherein the sub bandgap energy is only present when nitrogen is a component of the thin film.

18. The method of claim 17, wherein the sub bandgap state corresponds to a set of nitrogen related states in the thin film.

19. A method of analyzing a thin film, the method comprising:
obtaining a substrate and a thin film disposed above the substrate, the thin film including an interfacial layer directly above the substrate, and a high-k layer directly above the interfacial layer;
determining a thickness of the thin film;
analyzing the thin film by using deep ultraviolet spectroscopy ellipsometry to cause reflected light and determining photon energy of the reflected light;
using a model to extract a set of bandgap energies from a set of results of the determined photon energy of the reflected light; and
determining a property of the thin film using at least one of said set of bandgap energies.

20. The method of claim 19, wherein the property of the thin film is a leakage current or a nitrogen content and wherein said one of said set of bandgap energies is a main bandgap energy or a sub bandgap energy respectively.
